(19) United States
(12) Patent Application Publication
(10) Pub. No.: US 2017/0080011 A1
(43) Pub. Date: Mar. 23, 2017

(54) USE OF NADH AND NMN IN PREPARATION OF DRUGS OR HEALTH-CARE PRODUCTS FOR TREATING PARKINSON'S DISEASE

(71) Applicant: BONTAC BIO-ENGINEERING (SHENZHEN) CO., LTD, Shenzhen (CN)

(72) Inventors: Rongzhao FU, Shenzhen (CN); Qi ZHANG, Shenzhen (CN)

(21) Appl. No.: 15/311,797

(22) PCT Filed: Dec. 3, 2015
(86) PCT No.: PCT/CN2015/096288

§ 371 Date: Nov. 16, 2016

(30) Foreign Application Priority Data
Mar. 16, 2015 (CN) 201510113593.7

(51) Int. Cl. A61K 31/7084; A61K 9/28; A61K 9/20

(52) U.S. Cl. CPC A61K 31/7084; A61K 9/20; A61K 9/28

(57) ABSTRACT

Disclosed is use of nicotinamide adenine dinucleotide (NADH) and β-nicotinamide mononucleotide (NMN) in the preparation of drugs or health-care products for treating Parkinson's disease, in which β-NADH and βs-NMN are produced enzymatically.

### BACKGROUND

Technical Field

The present invention relates to the technical field of drugs and health-care products, and particularly to use of NADH and NMN in the preparation of drugs or health-care products for treating Parkinson's disease.

Related Art

Parkinson's disease (PD), also known as paralysis agitans, is one of the most common neurodegenerative diseases. Epidemiology shows that the prevalence is 15-328 per 10 million people, and is about 1% in populations of >65 years of age; and the incidence rate is 10-21/100,000 population/year.

In the prior art, the drugs or health-care products for treating Parkinson's disease includes compound levodopa preparation, dopamine receptor agonists, monoamine oxidase inhibitors, anticholinergic agents and amantadine. Some patients may consider the ablative procedure or deep brain stimulation (DBS). However, the existing drugs or health-care products are costive, which is hard to be accepted by the patients, and the effect still needs to be improved.

Therefore, there is still a need for improvement and development in the prior art.

### SUMMARY

### Technical Problem

In view of the disadvantages existing in the prior art, an objective of the present invention is to provide use of NADH and NMN in the preparation of drugs or health-care products for treating Parkinson's disease, so as to solve the problem of high cost and inadequate effect of existing drugs or health-care products for treating Parkinson's disease.

### Solution to the Problem

### Technical solution

The technical solution of the present invention is as follows.

Use of NADH and NMN in the preparation of drugs or health-care products for treating Parkinson's disease is provided, where

the β-NADH and β-NMN are used in the preparation of drugs or health-care products for treating Parkinson's disease.

In the use of NADH and NMN in the preparation of drugs or health-care products for treating Parkinson's disease, a single dose of the β-NMN is 0.1-500 mg/Kg body weight/day, and a single dose of the β-NADH is 0.01-50 mg/Kg body weight/day.

In the use of NADH and NMN in the preparation of drugs or health-care products for treating Parkinson's disease, a single dose of the β-NMN is 1-20 mg/Kg body weight/day, and a single dose of the β-NADH is 0.1-5 mg/Kg body weight/day.

In the use of NADH and NMN in the preparation of drugs or health-care products for treating Parkinson's disease, the drug or health-care product is in the form of tablets, capsules, granules, aqueous solutions, enteric-coated preparations, or injections.

In the use of NADH and NMN in the preparation of drugs or health-care products for treating Parkinson's disease, the drug or health-care product is in the form of tablets.

In the use of NADH and NMN in the preparation of drugs or health-care products for treating Parkinson's disease, the tablets are enteric-coated tablets.

### Beneficial Effect of the Invention

### Beneficial Effect

In the present invention, a new drug or health-care product is provided, which is prepared with cost-effective and high-purify β-NADH and β-NMN produced enzymatically in vitro, for treating Parkinson's disease. The β-NADH and β-NMN are used as active ingredients in the preparation of drugs or health-care products for treating Parkinson's disease. Specific dosage depends on severity of the disease, route of administration, and other relevant factors. β-NADH and β-NMN are substances present in cells of organisms, which are highly safe as a drug or health-care product. Moreover, the NMN is a monomeric molecule and has obvious and stable efficacy.

### DETAILED DESCRIPTION

The present invention provides use of NADH and NMN in the preparation of drugs or health-care products for treating Parkinson's disease. To make the objective, technical solution, and effect of the present invention clearer, the present invention is described in further detail below. It should be understood that specific embodiments described herein are merely illustrative of, instead of limiting the present invention.

Nicotinamide adenine dinucleotide (NADH) is a coenzyme transferring protons (more precisely hydrogen ions), and involved in many metabolic reactions of cells. Nicotinamide adenine dinucleotide includes reduced nicotinamide adenine dinucleotide (NADH), which is also known as reduced coenzyme I; and oxidized nicotinamide adenine dinucleotide (NAD), which is also known as oxidized coenzyme I.

NADH is a fundamental redox coenzyme, which is key to both the respiration and the photosynthesis.

β-nicotinamide mononucleotide (NMN) is a biochemical substance present in cells of organimsm, and is one of the precursors for synthesizing nicotinamide adenine dinucleotide (also known as coenzyme I) in cells.

At present, NADH and NMN can be produced through fermentation with yeasts or chemical synthesis, or produced enzymatically in vitro. Chemical synthesis has the disadvantage of high cost and production of chiral compound, and both the NADH and NMN fermented with yeasts contain an organic solvent residue. In contrast, the NADH and NMN produced enzymatically in vitro contain no organic solvent residue, have no problem of chirality, and are isoforms of the β-NADH and β-NMN in organisms. The β-NADH and β-NMN produced enzymatically in vitro are cost effective.

In the present invention, β-NADH and β-NMN produced enzymatically (in vitro) are used in the preparation of drugs or health-care products for treating Parkinson's disease. β-NADH and β-NMN are substances present in cells of organisms, which are highly safe as a drug or health-care product. Moreover, the NMN is a monomeric molecule and has obvious and stable efficacy.

A single dose of β-NMN is 0.1-500 mg/Kg body weight/day, and a single dose of β-NADH is 0.01-50 mg/Kg body weight/day. Preferably, a single dose of β-NMN is 1-20 mg/Kg body weight/day, for example, 10 mg/Kg body weight/day; and a single dose of β-NADH is 0.1-5 mg/Kg body weight/day, for example 2 mg/Kg body weight/day. Specific dosage depends on severity of the disease, route of administration, and other relevant factors.

In the present invention, the drug or health-care product is in the form of tablets, capsules, granules, aqueous solutions, enteric-coated preparations, or injections.

The drug or health-care product is in the form of tablets, and preferably enteric-coated tablets.

In the present invention, β-NADH and β-NMN are used as part of the active ingredient in the preparation of drugs or health-care products for treating Parkinson's disease, and preferably as the sole active ingredient in the preparation of drugs or health-care products for treating Parkinson's disease.

### EXAMPLE 1

### Use of NADH and NMN in the Preparation of Drugs or Health-Care Products for Treating Parkinson's Disease

The NADH and NMN, together with pharmaceutically acceptable adjuvants, were prepared into various dosage forms by using conventional methods in the art of medicines and health-care products. When provided for oral administration, the NADH and NMN may be prepared into conventional solid preparations, for example, tablets, powders or capsules; and when provided for injection, the NADH and NMN may be prepared into injectable solutions.

(1) Weighing: 10 g β-NADH and 10 g β-NMN;

40 g D-mannitol; and 20 g microcrystalline cellulose were weighed.

(2) Mixing uniformly and tabletting: 0.5 g magnesium stearate was added, mixed uniformly and tabletted.

(3) Coating: The tablets were coated with 2.5 g seal coating and 8 g enteric coating.

(4) The tablets were packaged into a pharmaceutical composition.

### EXAMPLE 2

### Clinical Data Regarding Pharmaceutical Compositions Containing β-NADH and β-NMN in the treatment of Parkinson's disease

Diagnostic Criteria of Parkinson's Disease

1.1 Materials: enteric-coated tablets containing pharmaceutical compositions of β-NADH and β-NMN,

1.2 Patients included and clinical manifestation: a total of 100 patients with

Parkinson's disease, aged over 60 years. Main manifestation: tremor, slow movement, abnormal posture and gait. The Parkinson's syndrome caused by encephalitis, cerebrovascular disease, poisoning, and trauma were excluded, and distinction was made between it and hysterical and dystonia tremor.

1.3 Administration method: The tablets obtained in Example 1 were dissolved in water, and administered to the patients. Two tablets were taken every morning on an empty stomach. 30 days were a course of treatment, and 3 courses of treatment were employed.

Criteria for Efficacy Evaluation

Obvious effectiveness: The symptoms disappear, and the patient returns to be healthy normally.

Improvement: The symptoms are improved, and the patient returns to be healthy gradually.

Ineffectiveness: No obvious improvement

The therapeutic result is shown in Table 1

TABLE 1

| | | | Obvious | | | | | | |
|---|---|---|---|---|---|---|---|---|---|
| | | Duration | effectiveness | | Improvement | | | | Total |
| | Total | of | | Obvious | | Improvement | Ineffectiveness | | effective |
| | number | treatment | Case | effective | Case | rate | Case | Ineffective | rate |
| Type | of cases | (days) | number | rate (%) | number | (%) | number | rate (%) | (%) |
| PD | 100 | 90 | 89 | 89.0 | 7 | 7.0 | 4 | 4.0 | 96 |

PD: Patients with Parkinson's disease

It can be seen from the table above that the composition containing β-NADH and β-NMN of the present invention is obviously effective in treating Parkinson's disease, and the total effective rate can be up to 96%.

In summary, β-NADH and β-NMN are used as active ingredients in the preparation of drugs or health-care products for treating Parkinson's disease in the present invention. The composition containing β-NADH and β-NMN is obviously effective in treating Parkinson's disease.

It should be understood that the use of the present invention is not limited to those exemplified above. Modifications or changes may be made by those ordinarily skilled in the art based on the disclosure above, which are all contemplated in the protection scope of the present invention as defined by claims below.

What is claimed is:

1. Use of NADH and NMN in the preparation of drugs or health-care products for treating Parkinson's disease, wherein the β-NADH and β-NMN are used in the preparation of drugs for treating Parkinson's disease, and the β-NADH and β-NMN are produced enzymatically.

2. The use of NADH and NMN in the preparation of drugs or health-care products for treating Parkinson's disease according to claim 1, wherein a single dose of the β-NMN is 0.1-500 mg/Kg body weight/day, and a single dose of the β-NADH is 0.01-50 mg/Kg body weight/day.

3. The use of NADH and NMN in the preparation of drugs or health-care products for treating Parkinson's disease according to claim 2, wherein the single dose of the β-NMN is 1-20 mg/Kg body weight/day, and the single dose of the β-NADH is 0.1-5 mg/Kg body weight/day.

4. The use of NADH and NMN in the preparation of drugs or health-care products for treating Parkinson's disease according to claim 1, wherein the drug or health-care product is in the form of tablets, capsules, granules, aqueous solutions, enteric-coated preparations or injections.

5. The use of NADH and NMN in the preparation of drugs or health-care products for treating Parkinson's disease according to claim 4, wherein the drug or health-care product is in the form of tablets.

6. The use of NADH and NMN in the preparation of drugs or health-care products for treating Parkinson's disease according to claim 5, wherein the tablets are enteric-coated tablets.
